(19) United States
(12) Patent Application Publication
(10) Pub. No.: US 2005/0277902 A1
(43) Pub. Date: Dec. 15, 2005

(54) Training inter-labial pad and inserting kit

(75) Inventors: Satoshi Mizutani, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/149,957

(22) Filed: Jun. 10, 2005

(30) Foreign Application Priority Data
Jun. 11, 2004 (JP) 2004-174864

(51) Int. Cl. A61F 13/15

(52) U.S. Cl. 604/385.17

(57) ABSTRACT

An inter-labial pad of a constitution tending to deposit a body fluid and of a structure tending to deform easily at the surface in contact with the inside of the labia when inserted is provided as a training inter-labial pad for training, as well as using an inserting kit having such a training inter-labial pad and indication means capable of checking the use thereof is provided, in which an external container of the inserting kit contains a training inter-labial pad for training the state of use thereof being recognizable easily after use and usual inter-labial pad are incorporated in individual wrapping containers together with a check sheet respectively.

### CROSS-REFERENCE TO RELATED APPLICATIONS

This application is based upon and claims the benefit of priority from Japanese Patent application No. 2004-174864 filed on Jun. 11, 2004, the entire contents of which is incorporated herein by reference.

### FIELD OF THE INVENTION

The present invention relates to a training inter-labial pad for training the use of an inter-labial pad and, more specifically, it relates to a training inter-labial pad for training devised such that even a beginner of an inter-labial pad can exactly understand the method of use by experiencing the actual use, and an insertion kit having the training inter-labial pad.

### RELATED ART

Inter-labial pads attached to female labia for absorbing menstrual blood have generally been known so far. Since such inter-labial pads are in close contact with a body, they prevent the menstrual blood from being diffused to sit at a wide area with the body, so that they are sanitary and clean. Further, they also have a feature of giving less psychological resistance during insertion compared with tampons which are inserted into a vagina.

As inter-labial pads having such feature described above, those of various structures have been developed and, for example, the pamphlet of International Laid-Open WO 02/100315 discloses an inter-labial pad, which is folded at a fold along the longitudinal center axis of the inter-labial pad such that parts of a back surface are opposed to each other and in which the parts of the back surface are joined at least at one joining portion to each other.

As the wrapping pattern of the inter-labial pad described above, those in which inter-labial pads are sealed in plurality and, further, a manual showing the way of use is provided in a wrapping container were sold on trial about from May, 2000 to May, 2001, in Wisconsin Eau Claire, USA (trade name of products: Envive manufactured by Procter & Gamble Co.) However, since a beginner of an inter-labial pad does not understand how to use the inter-labial pad, the beginner cannot detect the inserting position when she uses the inter-labial pad for the first time, so that the inter-labial pad deforms irregularly during trial intending for correcting the wearing position again and again and sometimes it can be put no more. Further, even when a manual is attached and packaged together, the beginner sometimes does not read the manual, can not surely understand the manual even after reading or, further, even after she can wear correctly by careful reading, she is not still sure for “correct wearing” and has to move about with anxious feeling during attachment.

### SUMMARY OF THE INVENTION

The present invention has been achieved in view of the foregoing subject and intends to provide an inter-labial pad capable of recognizing a correct inserting method and an inserting position of an inter-labial pad by actually experiencing the use of the inter-labial pad.

Further, the invention also intends to provide a wrapping pattern capable of confirming whether the inter-labial pad could be attached at a correct inserting position and by an inserting method after the insertion of the inter-labial pad.

The inventors of the present invention have made earnest studies for solving the subject described above and, as a result, have found that the subject can be solved by using an inter-labial pad having a surface which comes in contact with the inside of the labia and has a structure that enables to recognize easily the state of use after insertion as a training inter-labial pad, as well as by using an inserting kit having a training inter-labial pad and indication means capable of checking whether the inserted state is proper or not, and have accomplished the invention. More specifically, the invention provides the followings.

(1) A training inter-labial pad for training the use of an inter-labial pad to be inserted between labia, wherein fine unevenness is provided on a surface to face a body at least in a region to be in contact with the body when inserted.

According to the present invention, the surface to be in contact with a wearer's body has fine unevenness. Accordingly, a body fluid is involved to the region to be in contact with the body when the training inter-labial pad is inserted and the body fluid is tended to be deposited. Therefore, by merely attaching the training inter-labial pad only for several seconds, it can be confirmed at a glance whether the pad could be inserted in proper position by visually observing the stain of the body fluid.

Further, since not only menstrual blood but also other various body fluids are liable to be deposited on the surface, a wearer can conduct practice also in the period other than the menstrual period thereby capable of avoiding the risk that the menstrual blood stains shorts, etc. during practice if she performs practice during the menstrual period.

A sheet having an unevenness formed by fiber aggregate on the surface to be in contact with the body may be used, or a sheet applied with secondary fabrication for providing the unevenness may also be used for providing such a fine unevenness on the surface to be in contact with a body of the training inter-labial pad,

(2) A training inter-labial pad for training the use of an inter-labial pad to be inserted between labia, wherein a group of protrusions comprising a plurality of protrusions the shape of which changes depending on pressure of insertion applied to the inter-labial pad is provided to a surface to be in contact with a body to at least in a region to be in contact with the body when inserted.

According to the present invention, the surface shape of the training inter-labial pad is changed easily in accordance with the change of pressure. Therefore, the surface shape is changed in a region under the labial inner pressure in a state where the training inter-labial pad is inserted between the labia. Thus, a wearer can confirm whether the pad can be inserted into a correct inserting position by recognizing the deformed portion visually or by the sense of touch.

Further, since the inserted position can be confirmed merely by the change of the surface shape, the training inter-labial pad does not deprive as far as mucus in the labia and the inside of the labia is less injured even when the training inter-labial pad is inserted for several times.

The protrusion preferably has shape, size and pitch that can easily confirm the change of the shape instantaneously. Specifically, the shape is not restricted and includes a concave shape, convex shape, lattice shape or corrugated shape. The concave shape or convex shape provided with unevenness is partially preferred intermittently in order to understand easily the portion applied with a body pressure. Since the concave shape is deformed also relative to an external pressure from below, the convex shape is preferred among all. The convex shape is not restricted and may be circular, rectangular, square, trigonal, etc., and a rounded circular shape is preferred in view of a foreign-body sensation to a wearer. The length or the width is within a range from 1 to 20 mm, preferably, within a range from 3 to 10 mm and the pitch is within a range from 1 to 20 mm and, preferably, within a range from 3 to 10 mm.

The protrusion may be provided by embossing and, in a case where it is a film, the protrusion may be provided such that valves formed by applying an open pore forming process in a so-called funnel shape are turned upward.

Further, adhesives, etc. may also be provided to the lower part of the protrusion such that the protrusion, when crushed, can be fixed with no restoration. Thus, a wearer can distinctly recognize the portion of the surface shape where it has been deformed. While the type of the adhesives is not particularly restricted, pressure sensitive adhesives mainly comprising synthetic rubber such as a styrene-ethylene-butylene-styrene-block copolymer (SEBS), styrene-butadiene-styrene block copolymer (SBS), or styrene-isoprene-styrene block copolymer (SIS) is preferred since the tack is developed upon application of the labial inner pressure. Further, also the coating pattern is not restricted particularly, and spiral coating, controlled seam coating, coater coating, curtain coater coating, or spray gun coating can be used appropriately.

In a case where the training inter-labial pad is constituted with a surface side sheet disposed on the surface to be in contact with a body, a back side sheet situated on the side opposite thereto and an absorbent body put between the surface side sheet and the back side sheet, the group of protrusions may be provided not only on the surface side sheet but also on all the elements situated in the direction of the thickness, that is, the group of protrusions may be provide on the absorbent body and the back side sheet situated lower than the surface side sheet as well.

(3) A training inter-labial pad for training the use of an inter-labial pad to be inserted between labia, wherein an indicating element indicating a region to be in contact with the labia is disposed to a surface to be in contact with the body when inserted.

According to the invention, the indicating element appears on the surface of the training inter-labial pad. Therefore, a user can wear the pad in accordance with the indicating element, which facilitates the inserting operation.

“Indicating element” may be of a shape of a single line disposed along the longitudinal center axis in the inter-labial pad of a longitudinal shape as will be described in (5) below, or it may be disposed so as to override the longitudinal center axis. In a case where the indicating element is disposed so as to override the longitudinal center line, the indicating element is disposed laterally so as to intersect the longitudinal direction perpendicularly. Accordingly, in the forward of the training inter-labial pad disposed in the vicinity of a wearer's clitoris when inserted, the wearer can confirm the forward and backward inserting positions of the indicating element as a reference by observing the training inter-labial pad from above in a state of inserting the training inter-labial pad between the labia.

Further, “indicating element” may also be disposed near both ends of a longitudinal inter-labia pad. In this case, it can be confirmed whether the left-to-right inserting portion was correct or not by observing the body fluid deposited to the training inter-labial pad or the deformation state of the training inter-labial pad per se after insertion.

Further, “indicating element” may also be formed so as to model the shape of the labium minus pudendi. This enables to distinctly recognize the inserting position before wearing and it can be confirmed that whether the element was inserted in the profile after insertion or not.

In a case where the training inter-labial pad comprises a surface side sheet disposed on the surface to be in contact with a body, a back side sheet situated on the side opposite thereto and an absorbent body put between the surface side sheet and the back side sheet, the indicating element is not always disposed to the surface side sheet per se but it may also be disposed to the absorbent body situated to the lower surface of the surface side sheet or the back side sheet so long as it can be seen from the surface through the surface side sheet.

Examples of the indicating element include provision of unevenness by embossing or hauling of the surface side sheet, change of color by using a third member such as pigmented hot melt adhesives, incorporation of an ink, pigmented threads, etc.

(4) The training inter-labial pad according to (3), wherein the training inter-labial pad is substantially longitudinal shape having a longitudinal direction and a lateral direction, and the indicating element is a linear shape provided on the longitudinal center axis.

According to the invention, the indicating element constitutes a virtual line connecting the forward end and the backward end of the longitudinal center axis of the training inter-labial pad. Accordingly, the virtual line serves as a reference before inserting the pad so as to situate the pad to a pudendal slit, and the virtual line can be served as a reference for confirmation after insertion whether the training inter-labial pad has been inserted correctly or not.

(5) A training inter-labial pad for training the use of an inter-labial pad to be inserted between labia wherein a moistened region is provided on a surface to face a body in a region at least to be in contact with the body when inserted.

According to the invention, the surface of the training inter-labial pad is somewhat moistened. Accordingly, in a case where the training inter-labial pad is inserted, the surface easily fits the mucus in the labia, so that it less gives foreign-body sensation to a wearer also in the period other than the menstrual period and enables comfortable training for the insertion of the inter-labial pad.

(6) An inserting kit for insertion comprising the training inter-labial pad according to any one of (1) to (5) above and indication means related with a state of using the training inter-labial pad.

According to invention, the training inter-labial pad capable of experiencing the use of an inter-labial pad and the indication means capable of confirming whether the insertion of the training inter-labial pad is correct or not are integrated. Accordingly, by inserting the training inter-labial pad and comparing the stain of the body fluid deposited to the training inter-labial pad and the state of deformation with the indication means, it can be confirmed whether the pad could be inserted to a correct inserting position or not.

“Indication means” includes, for example, a printed matter such as a check sheet, and “state of use” can be expressed by various means such as illustrations, graphics, letters, etc. that can be compared by a wearer with the training inter-labial pad that the wearer used. The state of use preferably expresses not only the correct state of use but also a plurality of states of wrong insertion.

In a case where the state of use is expressed by “state of deposition of body fluid (that is to say stain of body fluid)”, it can be confirmed as to whether the stain is deviated either forward or backward or either leftward or rightward relative to the labia and the body, or whether the attached depth is sufficient or not. On the other hand, in a case where the state of use is expressed by “state of deformation”, it can confirm as to whether the inter-labial pad could be inserted in a state where the entire labia were completely opened or the inter-labial pad could be inserted in parallel with the longitudinal direction of the labia or not.

Further, in a case where a combinational sheet to be used in combination with the inter-labial pad is included in the inserting kit, it is possible to confirm as to whether the combinational sheet could be attached to a correct attaching position relative to the inter-labial pad, specifically, as to whether the training inter-labial pad and the combinational sheet are not deviated forward or backward and rightward or leftward by the indication of the indication means for the “stain of the body fluid” to the combinational sheet. Further, since “deformation state” of the combinational sheet is shown, it can be confirmed as to whether the combinational sheet could be attached by a correct attaching method, specifically, as to whether the both sides of the combinational sheet deform so as to cover the both sides of the inter-labial pad and hinder the absorption surface of the inter-labial pad.

The “inserting kit” may comprise only the training inter-labial pad and the indication means and, in addition, may also comprise a usual inter-labial pad and a combinational sheet. Particularly, in a case where it comprises both the training inter-labial pad and a usual inter-labial pad, it comprises both the inter-labial pads for use in training and for actual use.

As described above, according to the invention, since even a beginner of the inter-labial pad can preliminarily practice for the correct inserting position and inserting method of the inter-labial pad by using the training inter-labial pad, she can correctly attach the inter-labial pad easily even when it is for the first time.

Furthermore, by adapting the inserting kit such that the inter-labial pad and the indication means showing the state of use after insertion are combined in set, she can confirm as to “whether the pad could be inserted correctly or not” and obtain knowledge “how it can be inserted correctly” all at once.

### BRIEF DESCRIPTION OF THE DRAWINGS

FIG. 1 is a view showing a training inter-labial pad according to a first embodiment of the present invention;

FIG. 2 is a view showing a training inter-labial pad according to a second embodiment of the invention;

FIG. 3 is a view showing a check sheet according to the embodiment;

FIG. 4 is a view showing a modified example of the check sheet according to the embodiment;

FIG. 5 is a view showing a further modified example of the check sheet according to the embodiment;

FIG. 6 is a view showing a training inter-labial pad according to a third embodiment of the invention;

FIG. 7 is a view showing a deformed state of the training inter-labial pad according to the third embodiment of the invention;

FIG. 8 is a view showing a training inter-labial pad according to a fourth embodiment of the invention;

FIG. 9 is a view showing a state of attaching the training inter-labial pad according to the fourth embodiment of the invention;

FIG. 10 is a view showing an inserting kit according to an embodiment of the invention;

FIG. 11 is a view showing a modified example of the inserting kit according to the embodiment;

FIG. 12 is a view showing another modified example of the inserting kit according to the embodiment;

FIG. 13 is a view showing a further modified example of the inserting kit according to the embodiment;

FIG. 14 is a view showing an individual wrapping container for a training inter-labial pad according to the embodiment.

### DESCRIPTION OF THE SYMBOLS

| | |
|---|---|
| 1, 41, 51 | training inter-labial pad |
| 2, 42, 52 | surface side sheet |
| 10, 20, 30, 62 | check sheet |
| 43 | protrusion |
| 53 | virtual line |
| 54 | slit portion of a pubic mound |
| 60A to 60D | inserting kit |
| 61, 61B | training inter-labial pad individual wrapping body |
| 61A, 64A | intermediate container |
| 63 | external container |
| 64 | inter-labial pad individual wrapping body |
| 65 | tape |
| 66 | individual wrapping container |

### DESCRIPTION OF THE PREFERRED EMBODIMENT

The present invention is to be described by way of each of embodiments with reference to the drawings. In the description for the following embodiments, identical constituent factors carry same reference numerals for which the descriptions are to be omitted or simplified.

<Training Inter-Labial Pad>

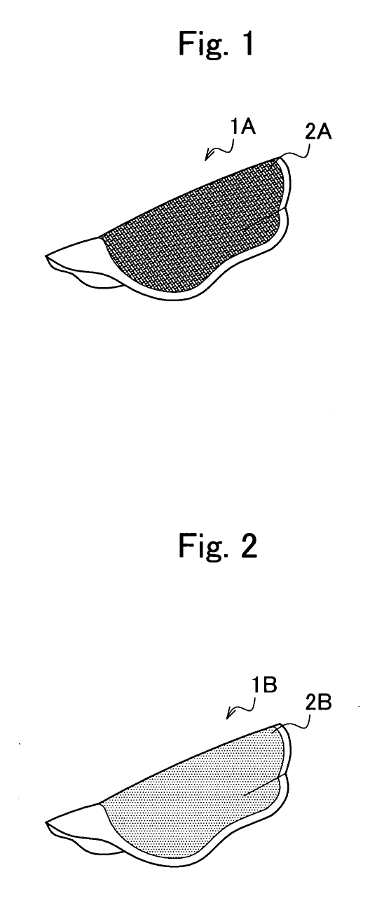

At first, first and second embodiments of the training inter-labial pad according to the invention are to be described. FIG. 1 is a view showing a training inter-labial pad 1A according to the first embodiment of the invention having a sheet comprising fibers constituting fine unevenness in which a body fluid tends to be deposited on the side of the surface thereof and FIG. 2 is a view showing a training inter-labial pad 1B according to the second embodiment of the invention having a sheet applied with secondary embossing such that a body fluid tends to be deposited on the side of the surface thereof.

FIG. 1 shows the training inter-labial pad 1A in which fine unevenness is formed due to the structure of a fiber aggregate on the surface of a surface side sheet 2A.

The training inter-labial pad 1 B shown in FIG. 2 is also provided with fine unevenness on a surface side sheet 2B, but different from FIG. 1 in that the unevenness is provided to the sheet by applying secondary fabrication.

[Surface Side Sheet]

Materials usable for the surface side sheet include non-woven fabrics obtained by a manufacturing method such as point bonding, through air and spun lacing alone or as a composite form. Further, they also include, for example, melt blown fibers constituted with ultra fine fibers with an extremely small inter-fiber distance, a film applied with fine unevenness on surface by passing along rolls having fine unevenness upon film formation, a sheet applied with fine protrusions by pin embossing or needling, or a sheet laminated to a fiber layer and treated by water jet fabrication on the side of the fiber layer to have a plurality of fine protrusions. The sheets may also be formed with permeable apertures.

Then, description is to be made specifically to a case of providing the fine unevenness on a film surface by a roll having fine unevenness. The roll having fine unevenness includes, for example, those having fine unevenness by flame spraying metal pieces with a grain size of 5 to 500 μm to the surface, in which the unevenness can be transferred to the film. The fine unevenness on the film surface includes those having a height and a pitch each of 5 to 500 μm. By such roll fabrication, the body fluid can intrude into concave portions of the film having the height and the pitch within the range described above, and the body fluid tends to be caught on the surface side sheet easily.

Then, description is to be made specifically to a case of providing the fine protrusions. The fine protrusion formed by applying pin embossing to an apertured film has a height within a range from 0.1 to 2.0 mm and, preferably, within a range from 0.5 to 1.0 mm, and a pitch within a range from 0.3 to 5.0 mm, preferably, 1.0 to 3.0 mm. The range for “fine protrusion” shown here is larger than the range for “fine unevenness” shown previously, because the fine protrusions give hindrance making the deposited body fluid less diffusible. It is preferred that the body fluid is less diffusible as described above because the wearer can easily recognize the region in which the training inter-labial pad was attached.

Materials constituting the surface side sheet are selected from pulp, chemical pulp, rayon, acetate, natural cotton, polymeric absorbent, fibrous polymeric absorbent, synthetic fiber, etc. alone or as a mixture thereof. Among them, those to which the body fluid tends to be caught but in which the body fluid is less diffusible are preferred because the wear can easily recognize the region to which the training inter-labial pad was attached. Specifically, in a case where the skin-contacting side of the surface side sheet comprises a fiber aggregate, finer fiber diameter is preferred since the body fluid more tends to be caught. More specifically, the fiber denier is within a range from 0.05 to 3.3 dtex and, preferably, within a range from 0.1 to 2.2 dtex. Referring to the manufacturing method, the range of the fiber denier shown previously can be obtained by melt blowing capable of spinning a resin into an ultra fine size, or spun lacing comprising divisional fibers capable of providing ultra fine fibers by dividing the fibers used under an external pressure such as water jet. Further, it is preferred that the synthetic fibers are mixed mainly in order to prevent diffusion of the body fluid.

“Divisional fibers” described above are to be described specifically. For the divisional fibers, those composite fibers comprising resins of two or more different ingredients and divided by mechanical impact shock or those formed by provisionally bonding ultra fine fibers into fiber bundles are used suitably. As the combination of resins for the divisional fibers, those of different releasing property or solvent solubility are used preferably and, for example, combinations such as of polyamide resin and polyester resin, polyamide resin and polyolefinic resin, polyester resin and polyolefinic resin, polyester resin and polyacrylonitrile resin, etc. are suitable. Among all, a combination of a polyester resin and a polyamide resin is preferred since it is divided easily by the impact of water jet. For the divisional fibers, in a case where the fibers are excessively fine, since they twine to the roll to lower the fiber-opening property upon fiber opening, it is preferred that the denier of the divisional fibers before division is within a range from 1.1 to 5.5 dtex and the denier of the ultra fine fiber caused by division is within a range about from 0.05 to 1.0 dtex. The cross sectional shape of the divided ultra fine fibers is preferably those of cross sectional shapes having larger fiber surface area such as elliptic, crescent or triangular shape since the body fluid tends to be caught easily. Further, the divisional fiber may be completely divided into each of ultra fine fibers individually, or a portion of the divisional fiber may be separated into fine fibers.

In view of the above, examples of the surface side sheet to which the body fluid tends to be caught and in which it is less diffusible and comprising a fiber assembly on the skin-contacting surface include spun laced non-woven fabrics formed by mixing from 100 to 50% of divisional fibers comprising a polyester resin and a polyamide resin in combination (fiber denier before division of 2.2 to 4.4 dtex, fiber denier after division of 0.1 to 0.5 dtex) and from 0 to 50% of rayon or acetate (fiber denier of 1.1 to 6.6 dtex), then conditioning them within a range from 20 to 50 g/m², and conditioning the thickness within a range from 0.3 to 1.00 mm.

<Check Sheet (Indication Means)>

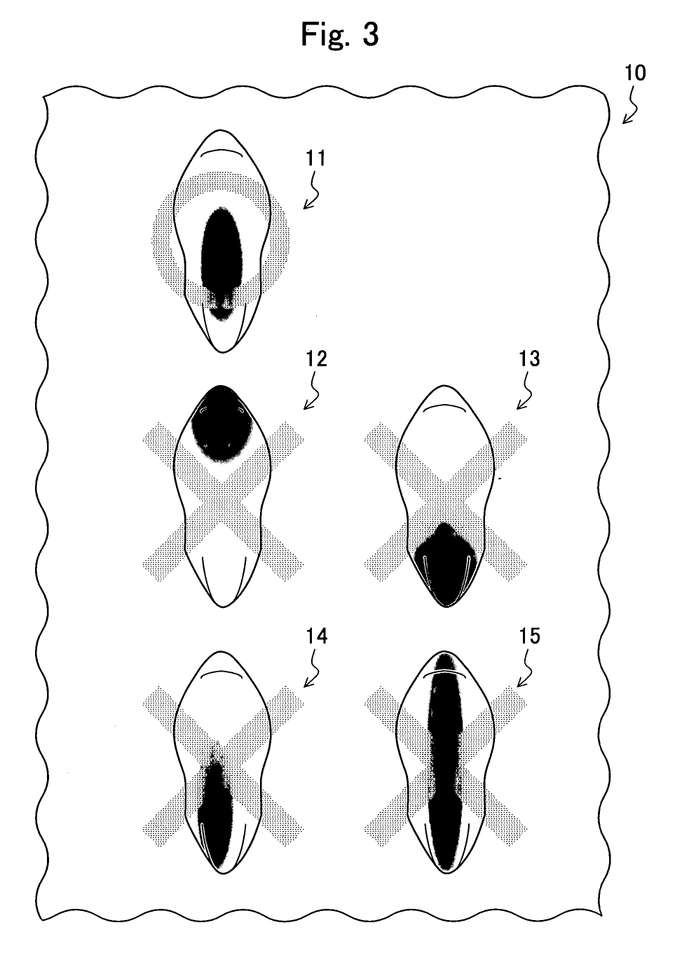

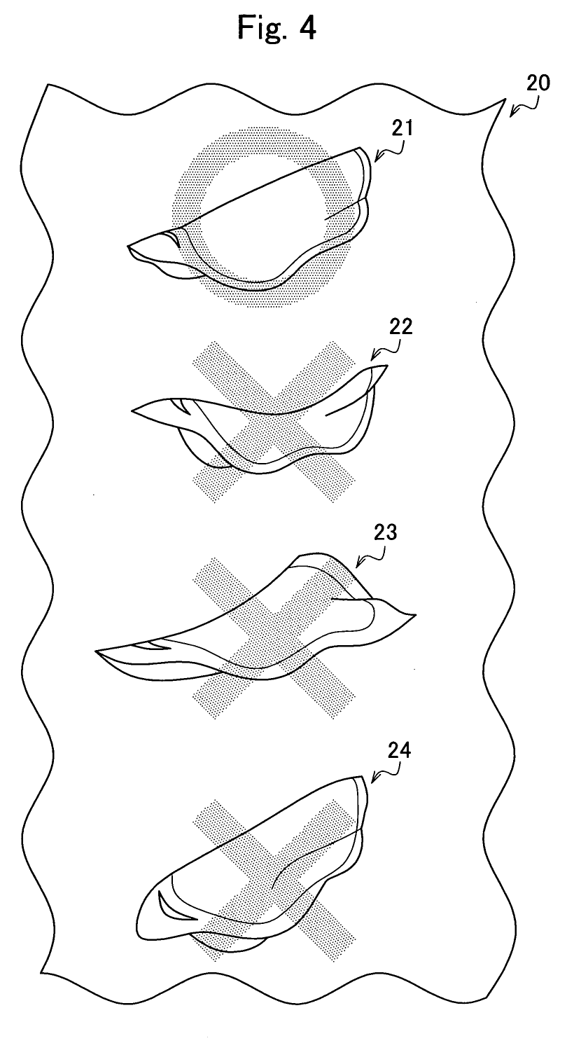

FIG. 3 is a view showing a check sheet 10 as an indication means which is a kind of a printed matter of illustrations 11 to 15 indicating body fluid deposition patterns for a training inter-labial pad and FIG. 4 is a view showing a check sheet 20 as an indication means which is a kind of a printed matter of illustrations 21 to 24 indicating deformation patterns for a training inter-labial pad.

Menstrual blood deposition patterns which are formed by inserting the training inter-labial pad are illustrated by illustrations 11 to 15 in the check sheet 10 shown FIG. 3. Among the illustrations 11 to 15, the illustration 11 shows a menstrual blood deposition pattern in a case of correct insertion and illustrations 12 to 15 show menstrual blood deposition patterns in a case of wrong insertion. Notes or advices are described such as 0 (correct) for the illustration 11, × (not correct) for the illustrations 12 to 15 and, further, “localized excessively backward, please insert a little forward” for illustration 12, “localized excessively forward, please insert a little backward” for illustration 13, “deviated rightward-to-leftward. Please insert in parallel with labia and along the center” for the illustration 14, and “inserting depth to labia is shallow, please insert while completely opening the entire labia” for the illustration 15. Accordingly, by comparing the training inter-labial pad that was attached by a wearer with each of the illustrations described in the check sheet 10 and confirming which illustration is more similar, it is possible to recognize whether the insertion made by the wearer was correct or not. Further, in a case where insertion was erroneous, by seeking an illustration of a pattern closer to the pattern of menstrual blood deposition, it is possible to correctly insert the pad in the next time in accordance with the advice described for the illustration.

The check sheet 20 shown in FIG. 4 is adapted such that when a wearer wears the training inter-labial pad and then detaches the pad, she can confirm whether the pad was inserted at a correct inserting position and by a correct inserting method or not by observing the deformation of the training inter-labial pad. Specifically, the check sheet 20 contains illustrations of inter-labial pads shown by references 21 to 24. Among them, the illustration 21 shows a deformation state in a case where the pad could be put at a correct inserting position and by a correct inserting method, and illustrations 22 to 24 show deformation states in a case where the pad was put at erroneous inserting positions and by erroneous inserting methods. Then, notes and advices are described for each of the illustrations such as ◯ for the illustration 21, × for the illustrations 22 to 24, and further, “pad is scarcely held between the inter-labia, please insert it while completely opening the entire labia” for illustration 22, “pad is not put at the forward, please insert it by completely opening the entire labia, particularly, at the forward labia” for illustration 23, and “pad is deviated rightward-to-leftward, please insert it in parallel with the labia and along the center” for illustration 24. Accordingly, by comparing the training inter-labial pad put by the wearer with the illustrations 21 to 24 described in the check sheet 20, it is possible to confirm whether the insertion made by her was correct or not. Further, in the case of erroneous insertion, by seeking an illustration of a pattern closer to the deformed state of the inter-labial pad for use in training of the wearer, it is possible to correctly insert the pad next time in accordance with the advice described for the illustration.

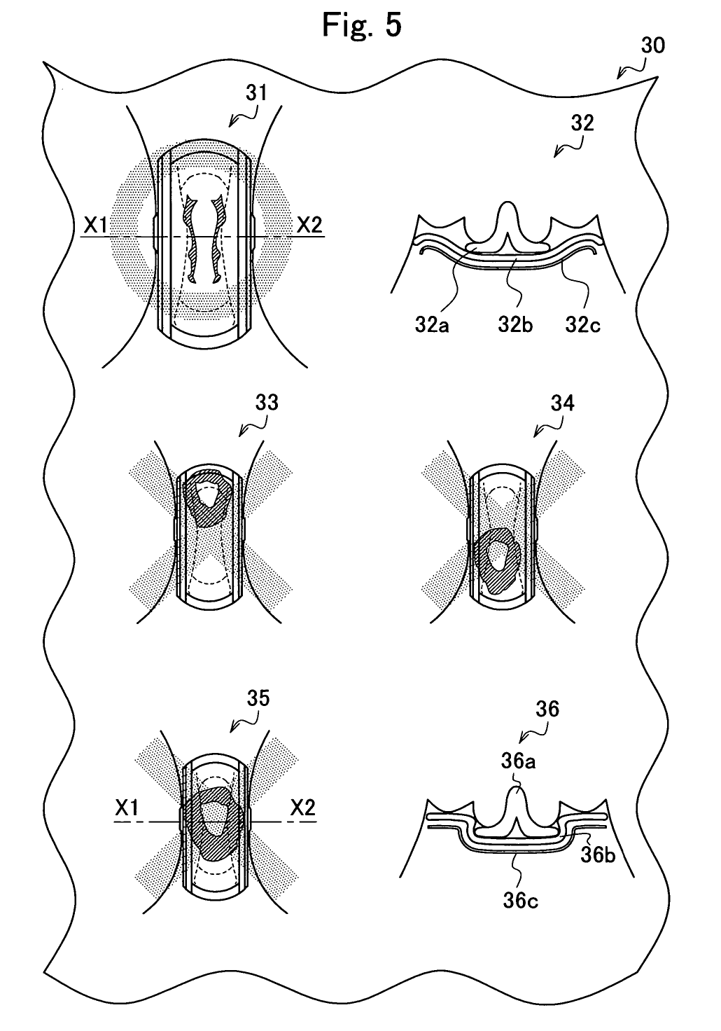

FIG. 5 is a view showing a check sheet 30 as an indication means, which is a printed matter expressing the stain of a body fluid deposited to the combinational sheet used in combination with the training inter-labial pad.

As shown in FIG. 5, the check sheet 30 shows illustrations 31 to 36 showing deposition patterns of a body fluid to the combinational sheet attached together with the training inter-labial pad. The illustration 31 shows a deposition pattern of a body fluid to the combinational sheet in a state where the training inter-labial pad (not illustrated) is attached at a correct inserting position and by a correct inserting method, and the illustration 32 is an illustration showing a cross section along X1-X2 of the illustration 31. The illustration 32 shows that the training inter-labial pad 32a is correctly put between the labia, under which the combinational sheet 32b and shorts 32c are snugly arranged. Further, the illustration 33 and the illustration 34 show the deposition pattern of a body fluid at erroneous positions and by erroneous methods. Then, notes and advices are described to the illustrations such as ◯ for the illustration 31, × for the illustrations 33 and the illustration 34, and “Pad is localized excessively backward, please insert it a little forward.” for illustration 33, and “Pad is localized excessively forward, Please insert it a little backward.” for illustration 34.

Further, the illustration 35 shows a state where the combinational sheet per se was attached in an erroneous state and notes and advices are described as “combinational sheet is attached under deformation. Please attach the sheet by pulling up in parallel while slightly closing feet when pulling up shorts”. The illustration 36 which is an illustration showing a cross section along X1-X2 of the illustration 35 expresses the state while the training inter-labial pad 36a is correctly inserted, the combinational sheet 36b is deformed on the shorts 36c.

With the check sheet 30 as described above, the attached state can be confirmed also for the combinational sheet. The check sheet 30 can be utilized not only for “attached state of the training inter-labial pad and the combinational sheet” described above but also for the confirmation of “the attached state of the combinational sheet in a case of using a usual inter-labial pad and a combinational sheet in combination”.

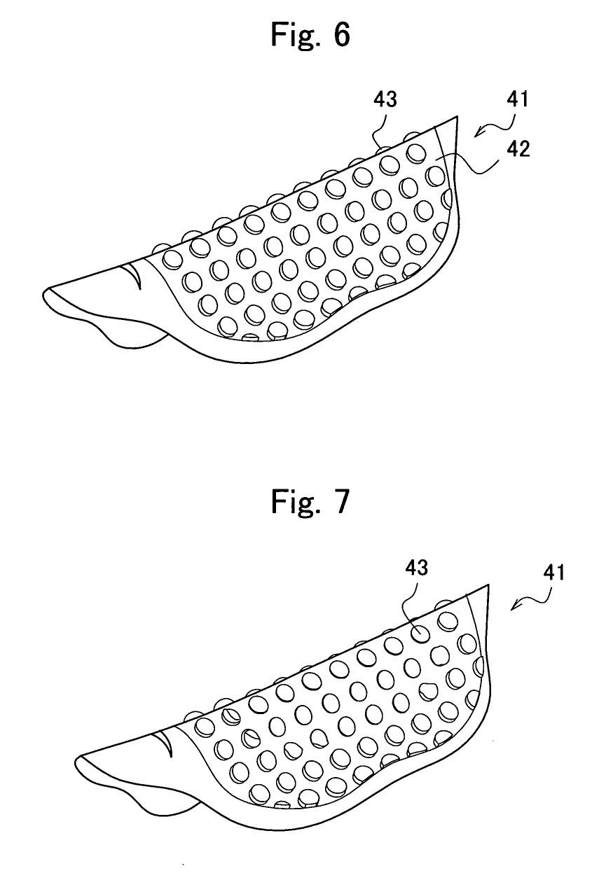

Then, a third embodiment of a training inter-labial pad according to the present invention is to be described. FIG. 6 is a view showing a training inter-labial pad 41 according to the third embodiment comprising a group of protrusions which are deformable by a pressure generated during wearing on a surface to be in contact with a body and FIG. 7 is a view showing a deformed state of the training inter-labial pad 41 in FIG. 6.

As shown in FIG. 6, the training inter-labial pad 41 comprises a group of protrusions having a plurality of protrusions 43 to a surface side sheet 42. Since the protrusion 43 is easily deformed depending on the change of the pressure, those portions in contact with a-body are concaved as shown in FIG. 7 when inserted. Accordingly, by observing the concaved portions, it is possible to confirm whether the inserting position was correct or not.

The protrusion 43 is formed, specifically, by embossing a spun laced non-woven fabric with an emboss pattern in which rounded rectangular convex bodies each of 5 mm length, 3 mm width and 3 mm height are arranged in a zigzag manner at 3 mm pitch, which is however not limitative.

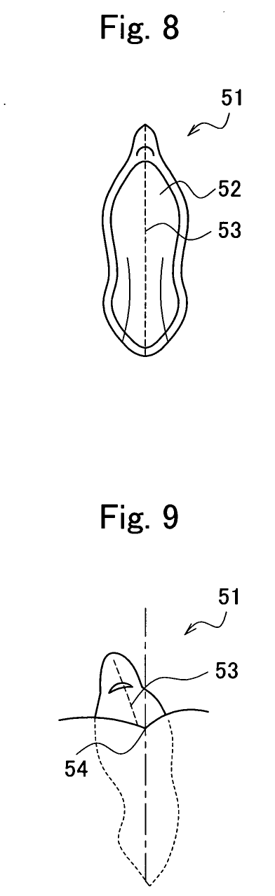

Then, a fourth embodiment of a training inter-labial pad according to the present invention is to be described. FIG. 8 is a view showing a training inter-labial pad 51 according to the forth embodiment having a virtual line as an indicating element showing an inserting position to a surface to be in contact with a body, and FIG. 9 is a view showing a state after inserting the training inter-labial pad 51 and before pulling up shorts.

A virtual line 53 as an indicating element connecting the forward end and a backward end of the longitudinal center axis is provided to the surface side sheet 52 of the training inter-labial pad 51 shown in FIG. 8. Accordingly, a wearer can insert such that the virtual line 53 is along a pudendal slit. Then, when viewing the training inter-labial pad 51 from above, the forward portion of the training inter-labial pad 51 can be recognized visually after inserting it as shown in FIG. 9, and a portion of the virtual line 53 can be confirmed. Then, since the virtual line 53 is deviated relative to a line extending from a slit portion 54 of a pubic mound situated at the center of the body (portion in communication from pudendum slit), it can be recognized that the inserting position is deviated.

Then, a training inter-labial pad (not illustrated) according to a fifth embodiment according to the invention is to be described.

According to this embodiment, the surface of the training inter-labial pad is in a somewhat moistened state. Therefore, the training inter-labial pad does not deprive as far as the mucus in the labia unnecessarily and training can be done also in other periods than the menstrual period.

“Somewhat moistened state” means a state at a humidity of official water content for cellulosic material (water content at a temperature of 20° C. and at a humidity of 65%), specifically, at 11% or more of official water content for rayon. Examples of humectants having a humidity of 11% or more of official water content include, glycerin, sodium hyaluronate, lecithin, squalane, urea, jojoba oil, myristyl alcohol, etc. and the surface can be kept in a moistened state by coating the humectant on fibers or films, or using the sheet containing the humectant on the surface side sheet.

Further, in a case where the humectant is not coated or contained to the surface side sheet, the check sheet or the like may have descriptions suggesting that a liquid such as tap water shall be coated on the surface side sheet, or menstrual blood remaining on a body is wiped off by wet tissue paper or the like, thereby somewhat moistening the body before a wearer inserts a training inter-labial pad.

<Inserting Kit>

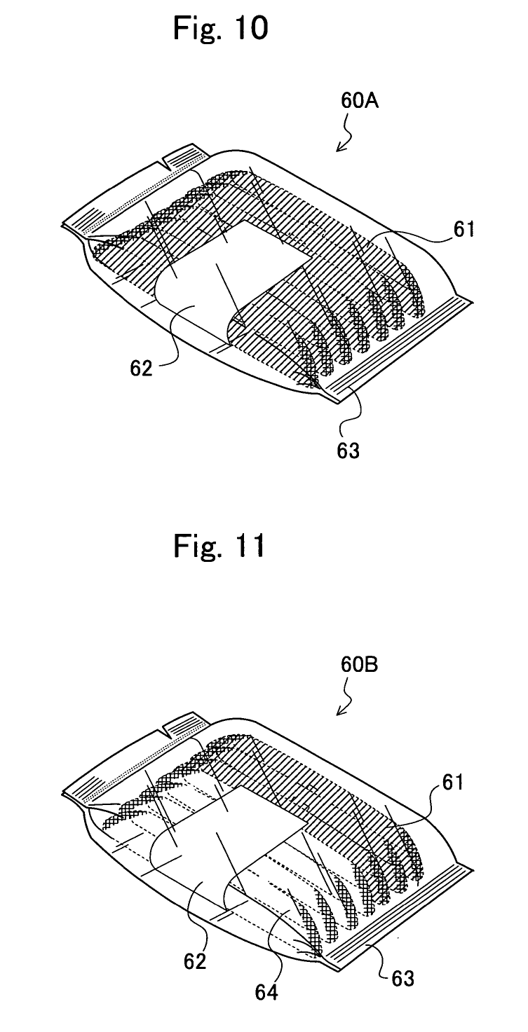

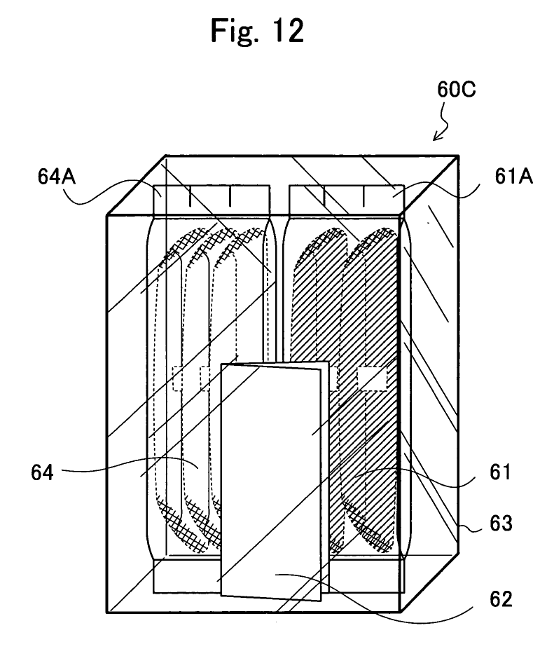

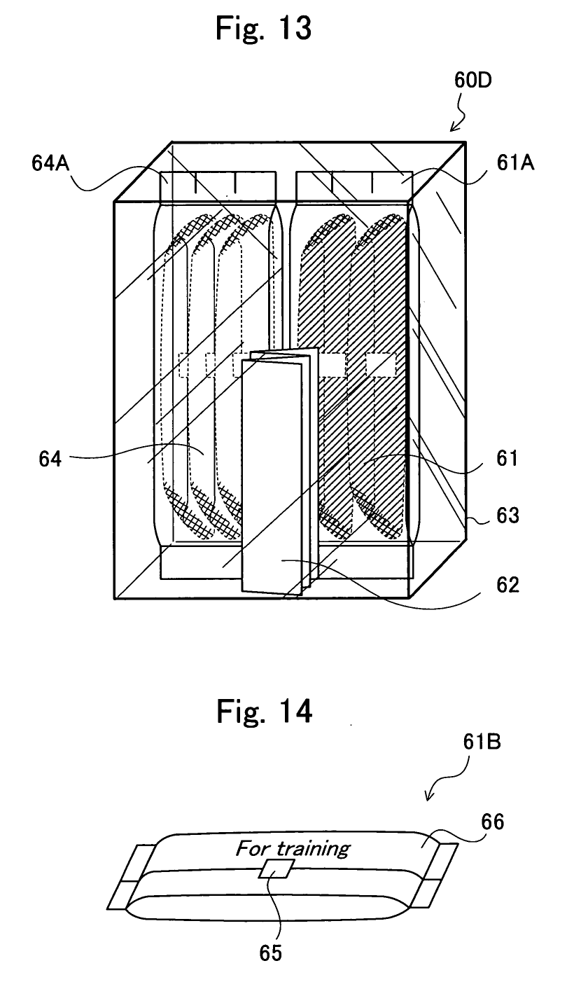

Then a first embodiment of an inserting kit according to the present invention is to be described. FIG. 10 is a view showing an inserting kit 60A in which training inter-labial pad individual wrapping bodies 61 each incorporating a training inter-labial pad and a check sheet 62 are contained in one external container 63. FIG. 11 is a view showing an inserting kit 60B in which training inter-labial pad individual wrapping bodies 61, inter-labial pad individual wrapping bodies 64 each incorporating an inter-labial pad, and a check sheet 62 are contained in an external container 63. FIG. 12 and FIG. 13 are views showing inserting kits 60C, 60D in which training inter-labial pad individual wrapping bodies 61 and inter-labial pad individual wrapping bodies 64 are contained respectively in intermediate containers 61A and 64A, and further, together with a check sheet 62 in an external container 63. FIG. 14 is a view showing an individual wrapping container 66 for a training inter-labial pad. In FIG. 14, the training inter-labial pad is incorporated in the individual wrapping container 66 to constitute a training inter-labial pad individual wrapping body 61B

As shown in FIG. 10, training inter-labial pad individual wrapping bodies 61 in which training inter-labial pads are sealed respectively in the individual wrapping containers are sealed by the number of seven together with a check sheet 62 in an external container 63 to constitute an inserting kit 60A for use in training. Accordingly, the method of using the inter-labial pad can be exercised for several times, for example, by opening the seal for the individual wrapping container, taking out the training individual pad, using the pad and then checking the state of use by the check sheet 62 thereby occasionally confirming as to whether the insertion was correct or not.

Further, in FIG. 11, inter-labial pad individual wrapping bodies 64 that incorporate usual inter-labial pads are incorporated together with the training inter-labial pad individual packages 61 together with the check sheet 62 in an external container 63, to form an inserting kit 60B. Accordingly, when a wearer carries about the inserting kit 60B, she can perform both the training for the inter-labial pad and actual use.

Further, FIG. 12 shows an inserting kit 60C comprising an external container 63 incorporated with training inter-labial pad individual wrapping bodies 61 by the number of two, inter-labial pad individual wrapping bodies 64 by the number of three contained in intermediate containers 61A and 64A respectively, and further containing a check sheet 62. Accordingly, when a wearer intends to conduct training, she only has to take out the intermediate container 61A in the sealed state and the check sheet 62, On the other hand, when she intends to use a usual inter-labial pad not for training, she only has to take out the intermediate container 64A in the sealed state, so that they can be selected optionally.

A combinational sheet to be used in combination with the training inter-labial pad or the inter-labial pad may be packed in the same package to the inside or the outside of the external container. Further, a portion for the content of the check sheet may be printed also to the individual wrapping container for the combinational sheet or the intermediate container.

The check sheet may be unfolded as shown in FIG. 10 and FIG. 11, or may be folded at the center as shown in FIG. 12, or may be folded several times as shown in FIG. 13. Further, it may be not a continuous sheet but may comprise plural sheets. Further, a portion for the content of the check sheet may be printed also to the individual wrapping container, the intermediate container or the external container. The check sheet may not always be contained in the external container but may be adhered to the outer surface thereof by means of adhesives, etc.

In a case where both the training inter-labial pads and the inner-labial pads are contained in the external container, it is preferred that the individual wrapping body incorporating the training inter-labial pad can be distinguished easily from the individual wrapping body incorporating the inter-labial pad. It is preferred, as shown in FIG. 14, that they can be distinguished, for example, by describing letters of “for training” in an individual wrapping container 66 in which a tape 65 is applied to a seal-opening for facilitating seal opening, as well as attaching illustration, by using a different color or by the absence or presence of the emboss pattern, or by the difference of the shape.

<Constitution for Training Inter-Labial Pad>

The constitution for training inter-labial pad preferably has such flexibility as giving less feels foreign-body sensation to a wearer as the beginner and thickness is preferably within a range from 1 to 10 mm and, more preferably, from 3 to 8 mm.

The shape is not particularly limited so long as the shape fits the female labia, such as an elliptic shape, hourglass shape or droplet shape. The total outer profile size is from 50 to 180 mm and, more preferably, from 80 to 120 mm in the longitudinal direction. Further, it is preferably from 30 to 100 mm and, more preferably, from 50 to 80 mm in the lateral direction. As an example of the constitution, it may comprise only the surface side sheet, or an absorbent body or a back side sheet may be disposed to the lower surface of the surface side sheet.

More preferably, it is desirable that a finger can be fixed to the back surface of the training inter-labial pad thereby further facilitating inserting. Specifically, a mini-sheet piece can be attached to the surface on the side opposite to the body-contacting surface thereby forming a finger insertion port in which a finger can be inserted.

<Absorbent Body>

In a case where an absorbent body is disposed to the training inter-labial pad, it is preferred to use absorbent body formed of pulp, chemical pulp, rayon, acetate, natural cotton, polymeric absorbent, fibrous polymeric absorbent, synthetic fibers, etc. alone or in admixture which are bulky and less defoamable and give less chemical stimulations. Specifically, they include non-woven fabric sheets having a basis weight per unit area of from 50 to 500 g/m² and a volume of from 1 to 20 mm formed by laminated fibers obtained by mixing from 95 to 60% of rayon or acetate selected form a range of a fiber denier of from 1.0 to 6.6 dtex, from 1 to 10% of a polymeric absorbent and from 4 to 30% of natural cotton, and then sheeting them by embossing. For the manufacturing method, they may be sheeted by an air laid method, spun lacing method, paper making method or the like and, further, applied with needling or embossing by passage between rolls of a dot-shape or lattice-like shape. In a case of embossing by using a dot-shaped emboss pattern, the emboss area ratio is preferably within a range from 0.3 to 60%.

[Back Side Sheet]

In a case where a back side sheet is further provided to the training inter-labial pad, those capable of preventing menstrual blood retained in the absorbent body from leaking to the outside of the inter-labial pad can be used. Further, by using the moisture permeable material, steaming during wearing can be decreased to reduce the uncomfortable feeling during wearing. Materials for the less water permeable sheet can include sheets of a thickness of from 15 to 60 μm formed by films comprising polyethylene, polypropylene, polyethylene terephthalate, polyvinyl alcohol, polylactic acid or polybutyl succinate, non-woven fabric, paper, as well as materials obtained by laminating them. Further, it may be an air permeable film obtained by mixing inorganic fillers into a resin and applying a stretching treatment. Specifically, they include films mainly comprising a low density polyethylene resin and conditioned to a basis weight per unit area within a range from 15 to 30 g/m² and, further, air permeable films obtained by open pore forming process controlled to an pore size within a range from 0.1 to 0.6 mm to have an open pore area percentage of from 10 to 30%. Examples of the non-woven fabric include spun bonded non-woven fabric, point bonded non-woven fabric, through air non-woven fabric, etc. which may be applied with a water repelling treatment. Among them, an SMS (spun bonded layer/melt blown layer/spun bonded layer) non-woven fabrics containing non-woven fabrics containing melt blown fibers constituted with ultra fine fibers and at an extremely small inter-fiber distance are preferred. In this case, it is preferably constituted such that the basis weight per unit area is from 5 to 15 g/m² for the spun bonded layer, from 1 to 10 g/m² for the melt blown layer, and from 5 to 15 g/m² for the spun bonded layer.

[Mini-Sheet]

In a case where the mini-sheet piece is attached to the training inter-labial pad, those having rigidity can be used for the mini-sheet. Further, by using moisture permeable materials, steaming during wearing can be decreased to reduce the uncomfortable feeling during wearing. Materials of such sheet include sheets of a thickness of from 15 to 60 μm formed by films comprising polyethylene, polypropylene, polyethylene terephthalate, polyvinyl alcohol, polylactic acid or polybutyl succinate, non-woven fabric, paper, as well as materials obtained by laminating them. Further, it may be an air permeable film obtained by mixing inorganic fillers into a resin and applying a stretching treatment. Examples of the non-woven fabric include spun bonded non-woven fabric, point bonded non-woven fabric, through air non-woven fabric, etc., which may be applied with a water repelling treatment. Among them, SMS non-woven fabrics containing melt blown fibers constituted with ultra fine fibers at an extremely small inter-fiber distance are preferred. In this case, the basis weight is preferably within a range from 5 to 15 g/m² for spun bonded layer, from 1 to 10 g/m² for melt blown layer, from 5 to 15 g/m² for the spun bonded layer. Further, the mini-sheet piece is preferably extended in accordance with the size of the wearer's finger, and an elastic material such as urethane or rubber may be used or embossing such as a corrugated shape may also be applied.

<Constitution of Combinational Sheet>

In the combinational sheet, adhesives for attaching to shorts are disposed and it is determined larger than the size at the back surface of the inter-labial pad in the state of wearing and it can receive and absorb to maintain menstrual blood overflown from the inter-labial pad during wearing.

The shape is not particularly limited so long as it fits the female body and shorts, for example, a rectangular shape, elliptic shape or hourglass shape, or a shape of a so-called wing for preventing deviation from the shorts. The total outer profile size is, preferably, from 100 to 500 mm and, more preferably, from 170 to 350 mm in the longitudinal direction and, preferably, from 30 to 200 mm and, more preferably, from 40 to 180 mm in the lateral direction.

An example of the constitution comprises a liquid permeable surface side sheet having a property of allowing a liquid to permeate therethrough as a cover member, a less liquid permeable back side sheet having a property of not substantially allowing a liquid to permeate therethrough as a cover member, an absorbent body disposed between the surface side sheet and the back side sheet and adhesives provided at the back surface of the back side sheet for preventing deviation from the shorts. Further, the sheet is sealed in a state three-folded or four-folded toward the inner surface when contained in an individual wrapping container, and a tape is provided to the seal opening of the individual wrapping container. Further, for easy releasing of the combinational sheet from the individual wrapping body, a releasing treatment such as with silicon may be applied on the inner surface of the individual wrapping body. Alternatively, a sheet subjected to a releasing treatment may be disposed between the combinational sheet and the individual wrapping body.

For conforming the combinational sheet more easily along the crotch shape, an emboss pattern copying the crotch shape, a so-called hinge, may be provided to the surface side sheet and the absorbent body. Further, in a case where the sheet applied with the releasing treatment is interposed between the combinational sheet and the individual wrapping body, the sheet includes, for example, paper formed by paper making process.

[Absorbent Body]

The absorbent body comprises, for example, pulp, chemical pulp, rayon, acetate, natural cotton, super absorbent polymer, super absorbent polymer fiber, or synthetic fiber alone or as a mixture thereof and it is preferably bulky, less deforming and giving less chemical stimulations. Specifically, it includes absorbent body having a basis weight per unit are of from 100 to 2000 g/m² and a volume of from 1 to 50 mm formed by mixing pulp within a range from 80 to 100% and a super absorbent polymer within a range from 20 to 0%, covering them with tissue paper and then sheeting them by embossing. Referring to the manufacturing method, sheeted materials formed by an air laid method, spun lacing method, paper making method, melt-blowing method, etc. and applied with needling, or embossing by passage between rolls having a dot-like, lattice-like or corrugated shape may be used. Further, for preventing the deformation of the absorbent body, the embossing area ratio is within a range from 10 to 100% and, preferably, from 30 to 80%.

<Individual Wrapping Container for Inter-Labial Pad>

For the materials of the individual wrapping containers for containing the inter-labial pad to form an individual wrapping body include those of a thickness of from 15 to 60 μm formed by films constituted with polyethylene, polypropylene, polyethylene terephthalate, polyvinyl alcohol, polylactic acid or polybutyl succinate, non-woven fabric, paper and materials obtained by laminating them. Specifically, they include films obtained by mixing a low density polyethylene resin within a range from 0 to 80% and a high density polyethylene within a range from 100 to 20%, and conditioning them to a basis weight per unit area within a range from 15 to 35 g/m². Further, they include those films applied with stretching for improving the resin orientation. Examples of the non-woven fabric can include, for example, spun bonded non-woven fabric, point bonded non-woven fabric and through air non-woven fabric, which may be applied with a water repelling treatment. Among them, SMS non-woven fabric containing melt blown fibers constituted with ultra fine fibers at an extremely small inter-fiber distance are preferred. In this case, the basis weight per unit area is, preferably, within a range from 5 to 15 g/m² for spun bonded layer, from 1 to 10 g/m² for melt blown layer, from 5 to 15 g/m² for spun bonded layer.

Further, it is preferred that the color of the menstrual blood absorbed by the inter-labial pad can be shielded and a pigment is mixed in a range from 0.2 to 10%, or an ink or the like may also be printed.

<Combinational Sheet>

A combinational sheet used together with the inter-labial pad is provided with adhesives for attachment to the shorts by bonding and it is sized at least larger than the size of the back surface of the inter-labial pad in a state of wearing, and those capable of receiving and absorbing to retain menstrual blood overflown from the inter-labial pad during wearing at the downward thereof are preferred.

<Shape>

The shape is not particularly limited so long as it has a shape fitting the shape of the female body and shorts, such as a rectangular shape, elliptic shape, hourglass shape or a shape attached with a so-called wing for preventing deviation from the shorts. The total size for the outer profile is from 10 to 500 mm and, more preferably, from 170 to 350 mm in the longitudinal direction. Further, it is, preferably, from 30 to 200 mm and, more preferably, from 40 to 180 mm in the lateral direction.

<Constitution>

As the constitution, it comprises a liquid permeable surface side sheet having a property of allowing a liquid to permeate therethrough as a cover member, a less liquid permeable back side sheet having a property of not substantially allowing the liquid to permeate therethrough as a cover member, an absorbent body interposed between the surface side sheet and the back side sheet, and adhesives disposed to the back face of the back side sheet for preventing the deviation from the shorts. Further, when it is contained in an individual wrapping container, it is sealed in a state being three-folded or four-folded toward the inner surface and the seal opening portion of the individual wrapping container is preferably provided with a tape for fixing. Further, in order to facilitate releasing of the combinational sheet from the individual wrapping container, a releasing treatment with silicon or the like may be provided to an inner surface of the individual wrapping container, or a sheet applied with a releasing treatment or the like may be disposed between the combinational sheet and the individual wrapping body.

<Absorbent Body>

The absorbent body comprises pulp, chemical pulp, rayon, acetate, natural cotton, polymeric absorbent, fibrous polymeric absorbent, synthetic fibers, etc. alone or as a mixture of them, and is preferably bulky, less deforming and giving less chemical stimulations. Specifically, the absorbent body includes those having a basis weight per unit area of 100 to 2000 g/m² and a volume of 1 to 50 mm formed by mixing from 80 to 100% of pulp and from 20 to 0% of absorbent polymer, covering them with tissue paper, and then sheeting them by embossing. As a method of manufacturing the absorbent body, those sheeted, for example, by an air laid method, spun lacing method, paper making method or melt blowing method and subjected to needling or embossing by passage between rolls of a dot-shape, lattice-shape or corrugated shape may also be used. Further, the emboss area ratio is, preferably, within a range from 10 to 100% and, more preferably, within a range from 30 to 80% also for preventing the deformation of the absorbent body.

For making the combinational sheet to easily conform the crotch shape, an emboss pattern copying the crotch shape, a so-called hinge, may be provided to the surface side sheet and the absorbent body. Further, in a case where the sheet applied with a releasing treatment is interposed between the combinational sheet and the individual wrapping container, the sheet can include paper prepared by a paper making method or the like.

<Example of Individual Wrapping Container for Inter-Labial Pad>

Materials for the individual wrapping container include sheet of a thickness from 15 to 60 μm formed by films constituted with polyethylene, polypropylene, polyethylene terephthalate, polyvinyl alcohol, polylactic acid or polybutyl succinate, non-woven fabric, paper or materials formed by laminating them. Specifically, the materials include those films formed by mixing a low density polyethylene resin with a range from 0 to 80%, and a high density polyethylene within a range from 100 to 20%, and conditioning them to a basis weight within a range from 15 to 35 g/m². Further, they also include films applied with stretching for improving the resin orientation. Examples of non-woven fabrics include, for example, spun bonded non-woven fabric, point bonded non-woven fabric and through air non-woven fabric, which may be applied with a water repelling treatment. Among them, SMS non-woven fabrics containing melt blown fibers constituted with ultra fine fibers at an extremely small inter-fiber distance are preferred. In this case, it is preferred that the basis weight is within a range from 5 to 15 g/m² for the spun bonded layer, from 1 to 10 g/m² for the melt blown layer, from 1 to 15 g/m² for the spun bonded layer.

Further, it is preferred that the individual wrapping container can shield the color of the body fluid absorbed by the inter-labial pad, and a pigment is mixed within a range from 0.2 to 10%, or an ink or the like may also be printed.

<External Container>

The material for the external container includes films constituted with polyethylene, polyethylene, polypropylene, polyethylene terephthalate, polyvinyl alcohol, polylactic acid or polybutyl succinate, non-woven fabric, paper, materials formed by laminating them, materials formed by vapor depositing aluminum to them, or plastics.

The invention can provide a training inter-labial pad for training the use of an inter-labial pad, or an inserting kit for recognizing correct use of the inter-labial pad by the training inter-labial pad and indication means related with the state of use thereof.

1. A training inter-labial pad for training the use of an inter-labial pad to be inserted between labia, wherein
fine unevenness is provided on a surface of the inter-labial pad to face a body at least in a region to be in contact with the body when inserted between the labia.

2. A training inter-labial pad for training the use of an inter-labial pad to be inserted between labia, wherein
a group of protrusions comprising a plurality of protrusions the shape of which changes depending on pressure of insertion applied to the inter-labial pad is provided on a surface of the inter-labial pad to be in contact with a body at least in a region to be in contact with the body when inserted between the labia.

3. A training inter-labial pad for training the use of an inter-labial pad to be inserted between labia, wherein
an indicating element indicating a region to be in contact with the labia is disposed on a surface of the inter-labial pad to be in contact with a body when inserted between the labia.

4. The training inter-labial pad according to claim 3, wherein
the training inter-labial pad is substantially longitudinal shape having a longitudinal direction and a lateral direction, and the indicating element is a linear shape provided on the longitudinal center axis.

5. A training inter-labial pad for training the use of an inter-labial pad to be inserted between labia, wherein
a moistened region is provided on a surface of the inter-labial pad to face a body at least in a region to be in contact with the body when inserted between the labia.

6. An inserting kit for insertion comprising the training inter-labial pad according to claim 1, and indication means related with a state of use of the training inter-labial pad.

7. An inserting kit for insertion comprising the training inter-labial pad according to claim 2 and indication means related with a state of use of the training inter-labial pad.

8. An inserting kit for insertion comprising the training inter-labial pad according to claim 3 and indication means related with a state of use of the training inter-labial pad.

9. An inserting kit for insertion comprising the training inter-labial pad according to claim 4 and indication means related with a state of use of the training inter-labial pad.

10. An inserting kit for insertion comprising the training inter-labial pad according to claim 5 and indication means related with a state of use of the training inter-labial pad.
